(19) United States
(12) Patent Application Publication
(10) Pub. No.: US 2023/0242915 A1
(43) Pub. Date: Aug. 3, 2023

(54) SMALL INTERFERING DEOXYRIBONUCLEIC ACID (SIDNA) AGAINST METALLO-BETA LACTAMASE GENE

(71) Applicants: Bahareh Cheshmenooshi, Kangavar (IR); Raoof Abdolmaleki, Kangavar (IR)

(72) Inventors: Bahareh Cheshmenooshi, Kangavar (IR); Raoof Abdolmaleki, Kangavar (IR)

(73) Assignees: Bahareh Cheshmenooshi, Kangavar (IR); Raoof Abdolmaleki, Kangavar (IR)

(21) Appl. No.: 17/884,034

(22) Filed: Aug. 9, 2022

Related U.S. Application Data
(63) Continuation-in-part of application No. PCT/IB2022/050039, filed on Jan. 4, 2022.
(60) Provisional application No. 63/133,252, filed on Jan. 1, 2021.

(51) Int. Cl. C12N 15/113

(52) U.S. Cl. CPC C12N 15/1137; C12N 2310/11; C12N 2310/113; C12N 2310/3517

(57) ABSTRACT

A small interfering deoxyribonucleic acid (siDNA) comprising a sense strand and an antisense strand that may be complementary to the sense strand. The sense strand may comprise the nucleic acid sequence set forth in SEQ ID NO: 1 and may be capable of hybridizing to a segment of a non-coding strand of class 1 integron metallo-beta-lactamase (blaIMP-1) gene. The antisense strand may comprise the nucleic acid sequence set forth in SEQ ID NO: 2 and may be capable of hybridizing to a segment of a coding strand of the blaIMP-1 gene.

### CROSS-REFERENCE TO RELATED APPLICATION

This application is a continuation-in-part of PCT Application PCT/IB2022/050039, filed on Jan. 4, 2022, entitled “SMALL INTERFERING DEOXYRIBONUCLEIC ACID (SIDNA) AGAINST METALLO-BETA LACTAMASE GENE,” which claims the benefit of priority from U.S. Provisional Patent Application Ser. No. 63/133,252, filed on Jan. 1, 2021, entitled “FLUORESCENCE SUPPRESSIVE DRUG COMPLEX OF RESISTANCE TO IMIPENEM AND MEROPENEM ANTIBIOTICS,” and which are both incorporated herein by reference in their entirety.

### TECHNICAL FIELD

The present disclosure generally relates to an exemplary small interfering deoxyribonucleic acid (siDNA) against an exemplary metallo-beta-lactamase (MBL) gene, and more particularly to an exemplary siDNA capable of silencing an exemplary class 1 integron metallo-beta-lactamase (blaIMP-1) gene.

### BACKGROUND

Beta-lactam antibiotics are one of the most commonly used antibiotics for treatment and management of microbial infections. Their use, however, has raised concerns about the growing phenomenon of bacterial resistance. There are several mechanisms by which bacteria may acquire resistance to beta-lactam antibiotics, one of which includes inactivation of beta-lactam antibiotics by producing beta-lactamases. In particular, beta-lactamases may catalyze hydrolysis of an amide bond in a beta-lactam antibiotic's ring, resulting in production of ineffective products. Beta-lactamases may be a frequent cause of resistance among certain Gram-positive species (such as Streptococcus pneumoniae) and among Gram-negative bacteria (such as Pseudomonas aeruginosa).

Silencing of the genes encoding beta-lactamases—at transcription or translation level—may be a promising strategy for treatment and management of infections caused by beta-lactam resistant bacteria. Small interfering ribonucleic acids (RNAs) and deoxyribonucleic acids (DNAs) are short nucleic acid molecules that may be used to interfere with expression of a certain gene at transcription or translation levels, through different mechanisms including—but not limited to—RNA-Induced Silencing Complex (RISC)-mediated mechanism, Clustered Regularly Interspaced Short Palindromic Repeats (CRISPR) system, and inhibition of DNA repair activities.

Thereby, considering the increasing health issues associated with the growth of resistance to beta-lactam antibiotics, there is need to develop new strategies and products for controlling and preventing resistance to such antibiotics.

### SUMMARY

This summary is intended to provide an overview of the subject matter of one or more exemplary embodiments, and is not intended to identify essential elements or key elements of the subject matter, nor is it intended to be used to determine the scope of the claimed implementations. Its sole purpose is to present some concepts of one or more aspects in a simplified form as a prelude to the more detailed description that is presented later. The proper scope of one or more exemplary embodiments may be ascertained from the claims set forth below in view of the detailed description below and the drawings.

In one general aspect, the present disclosure describes an exemplary small interfering deoxyribonucleic acid (siDNA). In an exemplary embodiment, an exemplary siDNA may comprise an exemplary sense strand and an exemplary antisense strand that may be complementary to an exemplary sense strand. In an exemplary embodiment, an exemplary sense strand may comprise an exemplary nucleic acid sequence set forth in SEQ ID NO: 1, and an exemplary antisense strand may comprise an exemplary nucleic acid sequence set forth in SEQ ID NO: 2.

In one or more exemplary embodiments, an exemplary antisense strand may be capable of hybridizing to an exemplary segment of an exemplary coding strand of class 1 integron metallo-beta-lactamase (blaIMP-1) gene. An exemplary coding strand of an exemplary blaIMP-1 gene may comprise an exemplary nucleic acid sequence set forth in SEQ ID NO: 3. An exemplary segment of an exemplary coding strand of an exemplary blaIMP-1 gene may comprise nucleotide 302 to nucleotide 325 of SEQ ID NO: 3. In one or more exemplary embodiments, an exemplary antisense strand may comprise an exemplary 6-Carboxyfluorescein (6-FAM) at the 5′ end of an exemplary antisense strand, and an exemplary tetramethylrhodamine (TAMARA) dye at the 3′ end of an exemplary antisense strand.

In one or more exemplary embodiments, an exemplary sense strand may be capable of hybridizing to an exemplary segment of an exemplary non-coding strand of an exemplary blaIMP-1 gene. An exemplary non-coding strand of an exemplary blaIMP-1 gene may comprise an exemplary nucleic acid sequence set forth in SEQ ID NO: 4. In an exemplary embodiment, an exemplary segment of an exemplary non-coding strand of an exemplary blaIMP-1 gene may comprise nucleotide 417 to nucleotide 440 of SEQ ID NO: 4. In an exemplary embodiment, an exemplary sense strand may comprise an exemplary protospacer adjacent motif (PAM). An exemplary PAM may include Cytosine 2 and Cytosine 3 (C2-C3) of SEQ ID NO: 1. In one or more exemplary embodiments, an exemplary sense strand may further comprise an exemplary 6-FAM at the 5′ end of an exemplary sense strand and an exemplary TAMARA dye at the 3′ end of an exemplary sense strand.

This Summary may introduce a number of concepts in a simplified format; the concepts are further disclosed within the “Detailed Description” section. This Summary is not intended to configure essential/key features of the claimed subject matter, nor is intended to limit the scope of the claimed subject matter.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which a presently preferred embodiment of the present disclosure will now be illustrated by way of example. It is expressly understood, however, that the drawings are for the purpose of illustration and description only and are not intended as a definition of the limits of the present disclosure. Embodiments of the present disclosure will now be described by way of example in association with the accompanying drawings in which:

FIG. 1A illustrates exemplary construct of an exemplary siDNA (small interfering deoxyribonucleic acid), consistent with one or more exemplary embodiments of the present disclosure;

FIG. 1B illustrates exemplary construct of an exemplary siDNA, consistent with one or more exemplary embodiments of the present disclosure;

FIG. 2 illustrates RNA expression profile in an exemplary transformed Pseudomonas aeruginosa with an exemplary siDNA (measured by real time reverse transcriptase polymerase chain reaction (RT-PCR)), consistent with one or more exemplary embodiments of the present disclosure;

FIG. 3 illustrates imipenem minimal inhibitory concentration (MIC) assay for an exemplary transformed Pseudomonas aeruginosa and an exemplary untreated Pseudomonas aeruginosa after 4, 18, and 36 h, consistent with one or more exemplary embodiments of the present disclosure;

FIG. 4 illustrates disc diffusion susceptibility testing for an exemplary transformed Pseudomonas aeruginosa and an exemplary untreated Pseudomonas aeruginosa (negative control) in the presence of an imipenem disc, consistent with one or more exemplary embodiments of the present disclosure;

FIG. 5A illustrates histological images of an exemplary excised lung tissue from an exemplary infected mouse with imipenem-resistant Pseudomonas aeruginosa which received neither of imipenem and/or an exemplary siDNA, consistent with one or more exemplary embodiments of the present disclosure; and

FIG. 5B illustrates histological images of an exemplary excised lung tissue from an exemplary infected mouse with imipenem-resistant Pseudomonas aeruginosa which received imipenem in combination with an exemplary siDNA, consistent with one or more exemplary embodiments of the present disclosure.

### DETAILED DESCRIPTION

In the following detailed description, numerous specific details are set forth by way of examples to provide a thorough understanding of the relevant teachings related to the exemplary embodiments. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be plain to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Provided herein is an exemplary small interfering deoxyribonucleic acid (siDNA) or a variant/analog thereof. One or more exemplary embodiments may be direct to an exemplary vector that may harbor an exemplary siDNA, an exemplary host cell carrying an exemplary siDNA, an exemplary nanocarrier carrying an exemplary siDNA, and/or an exemplary composition comprising an exemplary siDNA. In one or more exemplary embodiments, an exemplary siDNA may be capable of silencing or blocking or knocking down or knocking out the expression of an exemplary metallo-beta lactamase (MBL) gene. An exemplary MBL gene may result in microbial resistance to beta-lactam antibiotics. “Silencing,” “blocking,” “knockdown,” “knockout,” and equivalents thereof may refer to suppression and/or interruption and/or down-regulation of the expression of an exemplary gene (e.g., an exemplary MBL gene), at transcription or translation levels. In one or more exemplary embodiments, silencing, blocking, knockdown, and/or knockout of an exemplary gene may lead to reduction of gene expression or may stop/eliminate gene expression. For example, in one or more exemplary embodiments, silencing, blocking, knocking down, and/or knocking out of an exemplary gene may reduce expression of an exemplary gene by at least 10%, at least 20%, at least 30%, at least 40%, at least 50%, at least 60%, at least 70%, at least 80%, at least 90%, at least 95%, at least 98%, at least 98%, and at least 99%.

In an exemplary embodiment, an exemplary siDNA may be capable of silencing or blocking or knocking down or knocking out the expression of an exemplary MBL gene which may result in microbial resistance to beta-lactam antibiotics. Beta-lactam antibiotics may include a group of antibiotics used for management and treatment of bacterial infections. Beta-lactam antibiotics may have a 3-carbon and 1-nitrogen ring (beta-lactam ring) that may be highly reactive, and may include—but are not limited to—penicillin, cephalosporins, carbapenems, monobactams, and beta-lactamase inhibitors. There may be several mechanisms by which bacteria may acquire resistance to beta-lactam antibiotics, one of which may include inactivation of beta-lactam antibiotics by producing beta-lactamases. Beta-lactamases may include a diverse class of enzymes that may catalyze hydrolysis of a wide range of beta-lactam antibiotics including—but not limited to—carbapenems. Beta-lactamases may be a frequent cause of resistance to beta-lactam antibiotics in one or more exemplary Gram-positive species and one or more exemplary Gram-negative bacteria. Exemplary genes encoding beta-lactamases may be found on bacterial plasmids or bacterial chromosome. Beta-lactamases may be grouped into four exemplary classes, based on their primary sequence homology, including classes A, B, C, and D. In an exemplary embodiment, an exemplary siDNA may be capable of silencing or blocking or knocking down or knocking out the expression of an exemplary MBL (metallo-beta lactamase) gene. An exemplary MBL may be a class B beta-lactamase. Class B beta-lactamases may require a zinc for their activity and may catalyze hydrolysis of beta-lactam antibiotics through direct attack of a hydroxide ion to beta-lactam antibiotics. MBLs may include different types/families, including—but not limited to—IMPs, SPMs, VIMs, GIMs, and SIMs. In one or more exemplary embodiments, an exemplary siDNA may be capable of silencing an exemplary IMP gene. In an exemplary embodiment, an exemplary IMP gene may include—but is not limited to—class 1 integron metallo-beta-lactamase (blaIMP-1) gene. In an exemplary embodiment, an exemplary siDNA may be capable of silencing an exemplary blaIMP-1 gene. Thereby, an exemplary siDNA and/or exemplary compositions comprising an exemplary siDNA may be useful for treating microbial infections with bacteria including—but not limited to—Pseudomonas aeruginosa, Acinetobacter baumannii, Klebsiella pneumoniae, and Enterobacteriaceae.

An exemplary aspect may be directed to an exemplary siDNA that may be against blaIMP-1. In other words, in one or more exemplary embodiments, an exemplary siDNA may be capable of silencing or knocking down or knocking out an exemplary blaIMP-1 gene. “siDNA” may refer to exemplary short double stranded DNA molecules, 8-64 base pairs in length. Exemplary siDNA molecules may inhibit DNA repair activities by interfering with multiple DNA repair pathways and/or may interfere with DNA replication. Meanwhile, siDNA molecules may mimic DNA breaks and/or may interfere with recognition and repair of DNA damage induced on chromosomes. Furthermore, in prokaryotes and archaea, siDNAs may act as a foreign nucleic acid that—upon entering into an exemplary bacterial cell—may activate an exemplary adaptive immunity system known as CRISPR system. “CRISPR system” may refer to CRIPR complex—comprising a collection of CRISPR proteins and nucleic acid—that may result in at least one CRISPR-associated activity. “CRISPR activity,” or “CRISPR-associated activity” may refer to any activity associated with a CRISPR system. Examples of such activities include—but are not limited to—double-stranded cleavage, double-stranded nuclease, transcriptional activation, nickase, transcriptional repression, nucleic acid demethylation, nucleic acid methylation, and recombinase.

In an exemplary embodiment, an exemplary blaIMP-1 gene targeted by an exemplary siDNA may include an exemplary coding strand and an exemplary non-coding strand. An exemplary coding strand may have an exemplary nucleic acid sequence set forth in SEQ ID NO: 3 (nucleotide 114 to nucleotide 854 of GenBank: HM036079.1) and an exemplary non-coding strand may have an exemplary nucleic acid sequence set forth in SEQ ID NO: 4. In an exemplary embodiment, an exemplary coding stand of an exemplary blaIMP-1 gene may encode an exemplary blaIMP-1 enzyme that may have an exemplary amino acid sequence set forth in SEQ ID NO: 5. In one or more exemplary embodiments, an exemplary siDNA may be used to silence an exemplary blaIMP-1 gene in an exemplary bacteria that may be resistant to beta-lactam antibiotics, such as imipenem and meropenem. In one or more exemplary embodiments, an exemplary siDNA may silence an exemplary blaIMP-1 gene by activating an exemplary adaptive immunity system known as CRISPR system. CRISPR system may trigger a CRISPR complex to accomplish a CRISPR-associated activity including—but not limited to—double-stranded cleavage, double-stranded nuclease, transcriptional activation, nickase, transcriptional repression, nucleic acid demethylation, nucleic acid methylation, and recombinase. “CRISPR complex” may refer to CRISPR nucleic acids (e.g., RNA) and proteins that may associate with each other to form an aggregate that may have a CRISPR activity. In an exemplary embodiment, an exemplary siDNA may be used to silence an exemplary blaIMP-1 gene in Pseudomonas aeruginosa that may be resistant to beta-lactam antibiotics, such as imipenem and meropenem. In an exemplary embodiment, an exemplary siDNA may be capable of silencing an exemplary blaIMP-1 gene at transcription level (i.e., may prevent formation of an exemplary mRNA).

In one or more exemplary embodiments, an exemplary siDNA may have an exemplary sense strand and an exemplary antisense strand, wherein an exemplary antisense strand may be complementary to an exemplary sense strand. In an exemplary embodiment, an exemplary sense strand may comprise an exemplary nucleic acid sequence set forth in SEQ ID NO: 1 and an exemplary antisense strand may comprise an exemplary nucleic acid sequence set forth in SEQ ID NO: 2. In one or more exemplary embodiments, an exemplary sense strand may have an exemplary nucleic acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 99.5% sequence identity to SEQ ID NO: 1. In an exemplary embodiment, an exemplary sense strand may be at least 95 to 99.5% identical to SEQ ID NO: 1. In an exemplary embodiment, an exemplary sense strand may be at least 98% identical to SEQ ID NO: 1.

In one or more exemplary embodiments, an exemplary antisense strand may have an exemplary nucleic acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 99.5% sequence identity to SEQ ID NO: 2. In an exemplary embodiment, an exemplary antisense strand may be at least 95 to 99.5% identical to SEQ ID NO: 2. In an exemplary embodiment, an exemplary antisense strand may be at least 98% identical to SEQ ID NO: 2. “Identity” and “identical” may refer to a relationship between exemplary nucleic acid sequences of two or more polynucleotides that may be determined by comparing exemplary sequences. As appreciated by those skilled in the art, identity may also refer to an exemplary degree of relationship between the sequences calculated by determining number of matches between residues of two or more nucleic acid strings. Identity may be obtained by calculating the number of identical matches between two or more exemplary sequences (preferably the smaller one) by further considering gap alignments (if any) addressed by a computer program (e.g., algorithms) or a particular mathematical model. % Identity applied to two or more exemplary polynucleotide sequences may refer to the percentage of residues (i.e., nucleic acid residues) in an exemplary candidate nucleic acid sequence that may be identical to the residues in an exemplary second nucleic acid sequence after aligning exemplary sequences and gap alignment, if necessary, to achieve a maximum percent identity. In one or more exemplary embodiments, exemplary variants of an exemplary polynucleotide (i.e., an exemplary reference polynucleotide) may have at least 40% to at least 99%, but less than 100%, sequence identity to an exemplary reference polynucleotide.

In one or more exemplary embodiments, an exemplary antisense strand may be complementary to an exemplary sense strand (i.e., an exemplary sense and antisense strands may be complementary to one another). Being “complementary” may refer to an ability of an exemplary nucleic acid molecule to bind/hybridize to another exemplary nucleic acid molecule through Watson-Crick base pairing (between exemplary nucleotides of one exemplary nucleic acid molecule with exemplary nucleotides of another exemplary nucleic acid molecule). For example, in a canonical Watson-Crick base pairing, adenine (A) may form an exemplary base pair with thymine (T), and guanine (G) may form an exemplary base pair with cytosine (C) in DNA (in RNA, thymine (T) may be replaced by uracil (U)). It may be appreciated by one skilled in the art that “being complementary” may be used for an exemplary nucleic acid sequence that may be at least partially complementary to another exemplary nucleic acid sequence. Being complementary may also encompass exemplary nucleic acid duplexes that may be fully complementary, such that every nucleotide in one exemplary strand may be complementary to every corresponding nucleotide in another exemplary strand in all corresponding positions.

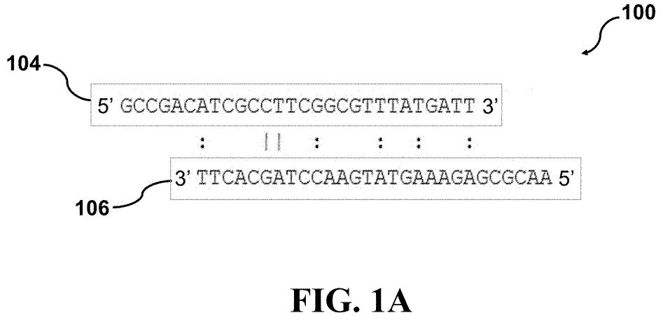

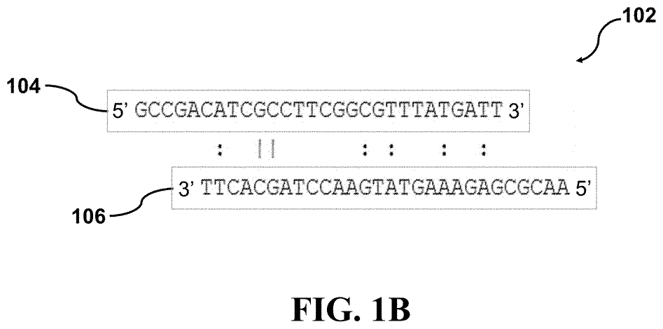

In an exemplary antisense strand may be partially complementary to an exemplary sense strand. In one or more exemplary embodiments, being partially complementary may refer to an exemplary state in which not all nucleotides are complementary to every nucleotide in another exemplary nucleic acid sequence in all corresponding positions. In one or more exemplary embodiments, being partially complementary may refer to a complementarity of at least 40%, at least 45%, at least 50%, at least 55%, at least 60%, at least 65%, at least 70%, at least 75%, at least 80%, at least 85%, at least 90%, at least 95%, at least 98%, at least 99%, and at least 99.5%. In one or more exemplary embodiments, an exemplary antisense strand of an exemplary siDNA may be at least 40%, at least 45%, at least 50%, at least 55%, at least 60%, at least 65%, at least 70%, at least 75%, at least 80%, at least 85%, at least 90%, at least 95%, at least 98%, at least 99%, and at least 99.5% complementary to an exemplary sense strand of an exemplary siDNA. For example, FIG. 1A illustrates exemplary construct 100 of an exemplary siDNA, consistent with one or more exemplary embodiments of the present disclosure. FIG. 1B illustrates exemplary construct 102 of an exemplary siDNA, consistent with one or more exemplary embodiments of the present disclosure. Referring to FIG. 1A and FIG. 1B, both of exemplary construct 100 and exemplary construct 102 may comprise exemplary sense strand 104 (having an exemplary nucleic acid sequence of SEQ ID NO: 1) and exemplary antisense strand 106 (having an exemplary nucleic acid sequence of SEQ ID NO: 2). Meanwhile, exemplary sense strand 104 and exemplary antisense strand 106 may have a complementarity of less than 100% (i.e., may not have full complementarity). For example, with further reference to FIGS. 1A-1B, exemplary sense strand 104 and exemplary antisense strand 106 may have at least 40% or at least 50% complementarity to each other.

In an exemplary embodiment, an exemplary antisense strand of an exemplary siDNA (e.g., exemplary antisense strand 106) may be capable of hybridizing to an exemplary segment of an exemplary coding strand of an exemplary blaIMP-1 gene. In an exemplary embodiment, an exemplary coding strand of an exemplary blaIMP-1 gene may have an exemplary nucleic acid sequence set forth in SEQ ID NO: 3. In an exemplary embodiment, an exemplary segment of an exemplary coding strand of an exemplary blaIMP-1 gene may comprise nucleotide 302 to nucleotide 325 of SEQ ID NO: 3. Thereby, in an exemplary embodiment, an exemplary antisense strand of an exemplary siDNA (e.g., exemplary antisense strand 106) may be capable of hybridizing to exemplary nucleotides 302 to 325 of SEQ ID NO: 3. In an exemplary embodiments, an exemplary antisense strand of an exemplary siDNA (e.g., exemplary antisense strand 106) may be partially complementary to an exemplary segment of an exemplary coding strand of an exemplary blaIMP-1 gene (e.g., exemplary nucleotides 302 to 325 of SEQ ID NO: 3). In one or more exemplary embodiments, an exemplary antisense strand of an exemplary siDNA (e.g., exemplary antisense strand 106) may have a complementarity of at least 40%, at least 45%, at least 50%, at least 55%, at least 60%, at least 65%, at least 70%, at least 75%, at least 80%, at least 85%, at least 90%, at least 95%, at least 98%, at least 99%, and at least 99.5% to an exemplary segment of an exemplary coding strand of an exemplary blaIMP-1 gene (e.g., exemplary nucleotides 302 to 325 of SEQ ID NO: 3).

In an exemplary embodiment, an exemplary sense strand of an exemplary siDNA (e.g., exemplary sense strand 104) may be capable of hybridizing to an exemplary segment of an exemplary non-coding strand of an exemplary blaIMP-1 gene. In an exemplary embodiment, an exemplary non-coding strand of an exemplary blaIMP-1 gene may have an exemplary nucleic acid sequence set forth in SEQ ID NO: 4. In an exemplary embodiment, an exemplary segment of an exemplary non-coding strand of an exemplary blaIMP-1 gene may comprise nucleotide 417 to nucleotide 440 of SEQ ID NO: 4. Thereby, in an exemplary embodiment, an exemplary sense strand of an exemplary siDNA (e.g., exemplary sense strand 104) may be capable of hybridizing to exemplary nucleotides 417 to 440 of SEQ ID NO: 4. In an exemplary embodiment, an exemplary sense strand of an exemplary siDNA (e.g., exemplary sense strand 104) may be partially complementary to an exemplary segment of an exemplary coding strand of an exemplary blaIMP-1 gene (e.g., exemplary nucleotides 417 to 440 of SEQ ID NO: 4). In one or more exemplary embodiments, an exemplary sense strand of an exemplary siDNA (e.g., exemplary sense strand 104) may have a complementarity of at least 40%, at least 45%, at least 50%, at least 55%, at least 60%, at least 65%, at least 70%, at least 75%, at least 80%, at least 85%, at least 90%, at least 95%, at least 98%, at least 99%, and at least 99.5% to an exemplary segment of an exemplary non-coding strand of an exemplary blaIMP-1 gene (e.g., exemplary nucleotides 417 to 440 of SEQ ID NO: 4).

In an exemplary embodiment, an exemplary sense strand of an exemplary siDNA may comprise an exemplary protospacer adjacent motif (PAM). For example, with further regards to FIGS. 1A-1B, exemplary sense strand 104 may comprise an exemplary PAM. In an exemplary embodiment, an exemplary PAM may comprise an exemplary Cytosine 2 and an exemplary Cytosine 3 (C2-C3) of exemplary sense strand 104 (i.e., C2-C3 of SEQ ID NO: 1). An exemplary PAM may comprise an exemplary short DNA sequence (e.g., 2-6 base pairs in length) disposed next to or after an exemplary target region in an exemplary nucleic acid molecule that may be intended to be targeted by an exemplary CRISPR system (e.g., to be cleaved by an exemplary CRISPR system), such as CRISPR-Cas9 and/or CRISPR-Cas3. An exemplary PAM may be required for an exemplary Cas nuclease (or may assist an exemplary Cas nuclease) to cut an exemplary target region and may, for example, be found 3-4 nucleotides downstream from an exemplary cut site.

In an exemplary embodiment, hybridization of an exemplary antisense strand of an exemplary siDNA (e.g., exemplary antisense strand 106) to an exemplary segment of an exemplary coding strand of an exemplary blaIMP-1 gene (e.g., exemplary nucleotides 302 to 325 of SEQ ID NO: 3), and/or hybridization of an exemplary sense strand of an exemplary siDNA (e.g., exemplary sense strand 104) to an exemplary segment of an exemplary non-coding strand of an exemplary blaIMP-1 gene (e.g., exemplary nucleotides 417 to 440 of SEQ ID NO: 4) may prevent DNA replication. Meanwhile, in an exemplary embodiment, an exemplary siDNA may be detected—as an exemplary foreign DNA—by an exemplary CRISPR system of an exemplary target bacteria (e.g., Pseudomonas aeruginosa) that may be resistant to beta-lactam antibiotics, such as imipenem and meropenem. In an exemplary embodiment, one or both of exemplary sense and antisense strands may be captured and integrated into an exemplary CRISPR locus/array of an exemplary target bacteria as new spacers (i.e., a new spacer may be generated for each of exemplary sense and antisense strands). An exemplary CRISPR locus may be transcribed and processed to produce mature CRISPR RNAs (crRNA). Each exemplary crRNA may be configured to encode a unique spacer sequence. Each exemplary crRNA may be associated with one or more exemplary effector proteins (i.e., exemplary Cas proteins) that may use crRNAs as exemplary guides to silence (e.g., degrade and/or cleave) genetic elements that may match an exemplary crRNA sequence. For example, in one or more exemplary embodiments, exemplary one or more effector proteins may use exemplary crRNAs to silence or block or knockdown or knockout (e.g., by degrading and/or cleaving) at least one of exemplary sense strand of an exemplary siDNA (e.g., exemplary sense strand 104), an exemplary antisense strand of an exemplary siDNA (e.g., exemplary antisense strand 106), an exemplary coding strand of an exemplary blaIMP-1 gene (or an exemplary segment of an exemplary coding strand of an exemplary blaIMP-1 gene), an exemplary non-coding strand of an exemplary blaIMP-1 gene (or an exemplary segment of an exemplary non-coding strand of an exemplary blaIMP-1 gene), or a combination thereof. In one or more exemplary embodiments, an exemplary siDNA may use an exemplary CRISPR system of an exemplary target bacterium (e.g., Pseudomonas aeruginosa) to silence or block or knockdown or knockout an exemplary blaIMP-1 gene (which may cause resistance to beta-lactam antibiotics, such as imipenem and meropenem) on bacterial chromosome.

In an exemplary embodiment, exemplary sense and antisense strands of an exemplary siDNA may further comprise an exemplary fluorescence dye at the 5′ end of an exemplary sense strand and the 5′ end of an exemplary antisense strand. Meanwhile, exemplary sense and antisense strands of an exemplary siDNA may also comprise an exemplary quencher at 3′ ends of exemplary sense and antisense strands. In an exemplary embodiment, an exemplary siDNA may comprise an exemplary 6-Carboxyfluorescein (6-FAM) at 5′ ends of exemplary sense and antisense strands (SEQ ID NOs: 1 and 2) and an exemplary tetramethylrhodamine (TAMARA) dye as an exemplary 3′-quencher at 3′ ends of exemplary sense and antisense strands (SEQ ID NOs: 1 and 2).

In an exemplary embodiment, an exemplary siDNA may be conjugated to and/or encapsulated in an exemplary nanocarrier including—but not limited to—polymeric nanoparticles, polymer conjugates, dendrimers, lipid-based carriers (including liposomes and micelles), carbon nanotubes, and gold nanoparticles. In an exemplary embodiment, exemplary polynucleotides disclosed herein may comprise an exemplary modification or segment (e.g., an exemplary sequence) that may lead to an exemplary desirable feature, such as increased stability, durability and affinity; and/or an additional moiety for subcellular targeting or tracking, such as a detectable label/tag, etc. In particular, exemplary modifications of exemplary polynucleotides may include, but are not limited to, modifications that may provide other chemical groups having or incorporating additional charge, hydrophobicity, polarizability, electrostatic interaction, hydrogen bonding, and fluxionality to the polynucleotide bases or to the polynucleotide as a whole. In one or more exemplary embodiments, exemplary modifications may provide nuclease-resistant oligonucleotides. Exemplary modifications may include one or more substituted inter-nucleotide linkages, altered bases, altered sugars, and exemplary combinations thereof. Exemplary modifications may further include, but are not limited to, 5′-position pyrimidine modifications, 2′-position sugar modifications, modifications at exocyclic amines, 8-position purine modifications, substitution of 5-bromo or 5-iodo-uracil, and 4-thiouridine substitution, phosphorothioate or alkyl phosphate modifications, methylations, backbone modifications, and unusual base-pairing combinations such as isoguanidine and isobases isocytidine. Exemplary modifications may further comprise exemplary 5′ and 3′ modifications, such as capping.

In one or more exemplary embodiments, an exemplary sense strand and an exemplary antisense strand may comprise non-natural nucleotide(s). Non-natural nucleotide may refer to an artificially-constructed nucleotide that may resemble, in chemical properties and/or structure, to an exemplary natural nucleotide. Examples of a non-natural nucleotide may include abasic nucleoside, arabinonucleoside, 2′-deoxyuridine, α-deoxyribonucleoside, β-L-deoxyribonucleoside, and exemplary glycosylated nucleosides. Exemplary glycosylated nucleosides may include glycosylated nucleosides having substituted pentose (2′-O-methylribose, 2′-deoxy-2′-fluororibose, 3′-O-methyl ribose, or 1′,2′-deoxyribose), arabinose, substituted arabinose sugar, substituted hexose, or alpha anomer. Exemplary non-natural nucleosides may further include an exemplary artificially-constructed base analog or an artificially chemically modified base. Examples of a base analog may include a 2-oxo(1H)-pyridin-3-yl group, a 5-substituted 2-oxo(1H)-pyridin-3-yl group, a 2-amino-6-(2-thiazolyl)purin-9-yl group, and a 2-amino-6-(2-oxazolyl)purin-9-yl group. Examples of the modified base may include modified pyrimidine (e.g., 5-hydroxycytosine, 5-fluorouracil and 4-thiouracil), modified purine (e.g., 6-methyladenine and 6-thioguanosine), and other heterocyclic bases.

In one or more exemplary embodiments, exemplary nucleic acid sequences disclosed herein may be prepared synthetically, preferably using an exemplary commercially available poly/oligo-nucleotide synthesizer. Exemplary methods of synthetic oligonucleotide synthesis include, but are not limited to, solid-phase oligonucleotide synthesis, and liquid-phase oligonucleotide synthesis.

### EXAMPLES

Hereinafter, one or more exemplary embodiments will be described in further detail with reference to examples. It will be obvious to a person having ordinary skill in the art that these examples may be for illustrative purposes only and are not to be interpreted to limit the scope of one or more exemplary embodiments.

### Example 1: Design and Synthesis of an Exemplary siDNA Targeting blaIMP-1 (Class 1 Integron Metallo-Beta-Lactamase) Gene

In this example, an exemplary siDNA was designed and synthesized for targeting an exemplary gene encoding blaIMP-1 that may be responsible for microbial resistance to beta-lactam antibiotics, such as imipenem and meropenem. In this example, an exemplary gene encoding blaIMP-1 (i.e., the coding strand of an exemplary blaIMP-1 gene) may comprise the nucleic acid sequence set forth in SEQ ID NO: 3 (nucleotide 114 to nucleotide 854 of GenBank: HM036079.1). An exemplary gene encoding blaIMP-1 may also be referred to as blaIMP-1 gene. In an exemplary embodiment, an exemplary blaIMP-1 gene may encode an exemplary blaIMP-1 enzyme having the amino acid sequence set forth in SEQ ID NO: 5 (also set forth in GenBank: ADH03007.1). The designed siDNA in this example may be configured to silence/block or knockdown or knockout expression of the blaIMP-1 gene.

In an exemplary embodiment, the designed siDNA may include a sense strand and an antisense strand, wherein the sense strand may be complementary (in particular, may be partially complementary) to the antisense strand. In an exemplary embodiment, an exemplary antisense strand of an exemplary siDNA may be at least 40%, at least 45%, at least 50%, at least 55%, at least 60%, at least 65%, at least 70%, at least 75%, at least 80%, at least 85%, at least 80%, at least 90%, at least 98%, at least 99%, and at least 99.5% complementary to an exemplary sense strand of an exemplary siDNA. In an exemplary embodiment, an exemplary sense strand may have the nucleic acid sequence set forth in SEQ ID NO: 1, and an exemplary antisense strand may have the nucleic acid sequence set forth in SEQ ID NO: 2. Two exemplary constructs of the designed siDNA, in this example, are shown in FIGS. 1A-1B (exemplary construct 100 and exemplary construct 102). With further reference to FIGS. 1A-1B, exemplary sense strand 104 and exemplary antisense strand 106 may have a complementarity of less than 100% (i.e., may not have full complementarity). For example, with further reference to FIGS. 1A-1B, exemplary sense strand 104 and exemplary antisense strand 106 may have at least 40% or at least 50% complementarity to each other.

In an exemplary embodiment, an exemplary antisense strand (SEQ ID NO: 2) may be complementary with nucleotide 302 to nucleotide 325 of an exemplary coding stand of the blaIMP-1 gene set forth in SEQ ID NO: 3. Meanwhile, an exemplary sense strand (SEQ ID NO: 1) may be complementary with nucleotide 417 to nucleotide 440 of an exemplary non-coding strand of the blaIMP-1 gene set forth in SEQ ID NO: 4. The designed siDNA in this example may further comprise a 6-Carboxyfluorescein (6-FAM) at the 5′ ends of exemplary sense and antisense strands (SEQ ID NOs: 1 and 2, respectively) and a tetramethylrhodamine (TAMARA) dye as a 3′-quencher at the 3′ ends of exemplary sense and antisense strands. In an exemplary embodiment, an exemplary sense strand (SEQ ID NO: 1) may further comprise a protospacer adjacent motif (PAM). In an exemplary embodiment, an exemplary PAM may include a Cytosine-Cytosine (CC) dinucleotide at the 5′ end/region of an exemplary sense strand. In this example, an exemplary CC dinucleotide may be disposed at positions/nucleotides 2 and 3 of an exemplary sense strand (SEQ ID NO: 1). Exemplary 6-FAM and TAMARA dyes may be used to follow and confirm: i) entry of an exemplary siDNA in an exemplary bacterial cell (e.g., Pseudomonas aeruginosa), ii) confirming that an exemplary siDNA has been targeted by CRISPR system of exemplary bacterial cells, iii) confirming that an exemplary siDNA may have been hybridized to an exemplary target gene (i.e., either an exemplary coding strand or an exemplary non-coding strand of the blaIMP-1 gene), and/or iv) confirming that an exemplary siDNA may have led to cleavage of an exemplary target gene (i.e., either an exemplary coding strand or an exemplary non-coding strand of the blaIMP-1 gene). 6-FAM may emit fluorescence light based on fluorescence resonance energy transfer (FRET) technique. In brief, in FRET technique, 6-FAM may be excited by being exposed to a 490 nm light. 6-FAM may further transfer its absorbed energy to an exemplary TAMARA dye that, in turn, may return 6-FAM to a ground state of energy and excite an exemplary TAMARA dye to an electronically excited state. An exemplary TAMARA dye may not emit or absorb any light in the electronically excited state. The amount of fluorescence loss by 6-FAM at 520 nm may be proportional to the amount of fluorescence gain by an exemplary TAMARA dye at 580 nm. Thereby, spatial proximity of 6-FAM to TAMARA may result in a high FRET efficiency which may be measured based on the relative fluorescence emissions of both 6-FAM and TAMARA dyes. On the other hand, spatial separation of 6-FAM from the TAMARA dye—e.g., due to the degradation of an exemplary siDNA by CRISPR system—may lead to breakdown of the FRET effect which may be detectable in fluorescence emission spectrum based on the ratio of red emission to green emission (R/G ratio).

In this example, exemplary sense and antisense strands (set forth in SEQ ID NOs: 1 and 2, respectively)—both comprising a 6-FAM at their 5′ end and a TAMARA dye at their 3′ end-were obtained synthetically. Exemplary sense and antisense strands (SEQ ID NOs: 1 and 2, respectively) were, then, annealed to one another. Formation of an exemplary duplex of siDNA (comprising exemplary sense and antisense strands) was subsequently confirmed by gel electrophoresis.

### Example 2: Transferring an Exemplary siDNA into Imipenem/Meropenem-Resistant Pseudomonas aeruginosa

In this example, the synthesized siDNA in “Example 1” (comprising exemplary sense and antisense strands set forth in SEQ ID NO: 1 and 2, respectively) was cloned into Pseudomonas aeruginosa bacteria harboring an exemplary blaIMP-1 gene (imipenem/meropenem resistance gene). To this end, first, competent Pseudomonas aeruginosa was prepared and stored at −70° C. to be used in further experiments. Competent cells may refer to microbial cells capable of taking up foreign nucleic acid molecules from their surroundings through a process known as transformation. In order to transfer/clone the prepared siDNA into the competent Pseudomonas aeruginosa bacteria, 100 μL of an exemplary siDNA (either being coated with chitosan polymer or being bare—i.e., being uncoated) with a concentration of about 1% w/v was added to 60 μL of the competent Pseudomonas aeruginosa culture in Lysogeny Broth (LB) medium, gently mixed, and kept at 0° C. for 45 min. Then, the mixture of an exemplary siDNA (either being bare or coated with chitosan polymer) with the culture of competent Pseudomonas aeruginosa was incubated at 37° C., for 20 sec, and immediately placed on ice. Then, 800 μL of Super Optimal broth (SOC) medium was added to the mixture of an exemplary siDNA (either being bare or coated with chitosan polymer) with the culture of competent Pseudomonas aeruginosa and incubated at 37° C., in a shaker incubator, for 24-30 h.

### Example 3: Evaluating the Effect of an Exemplary siDNA on the Expression of an Exemplary blaIMP-1 Gene in Pseudomonas aeruginosa

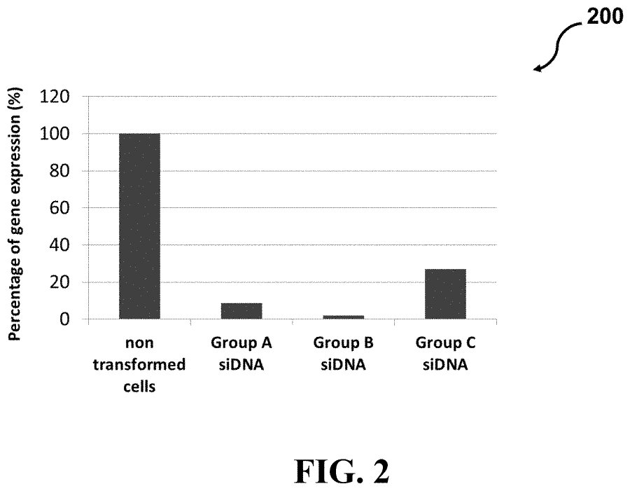

In this example, the effect of an exemplary siDNA—which was cloned into Pseudomonas aeruginosa bacteria in “Example 2”—on the expression of an exemplary blaIMP-1 gene was evaluated. In other words, the level of blaIMP-1 mRNA (messenger RNA) was measured in the transformed Pseudomonas aeruginosa bacteria with an exemplary siDNA in “Example 2”. For this purpose, the total RNA of an exemplary transformed Pseudomonas aeruginosa and an exemplary untreated Pseudomonas aeruginosa (as negative control) was extracted using a commercial RNA extraction kit, followed by conducting a real time RT-PCR (reverse transcriptase polymerase chain reaction) to determine the level of blaIMP-1 mRNA. In this example, 2-ΔΔCt method was used to determine differences in normalized expression of an exemplary blaIMP-1 gene (at transcription level) induced by an exemplary siDNA. FIG. 2 illustrates RNA expression profile 200 in an exemplary transformed Pseudomonas aeruginosa with an exemplary siDNA (measured by real time RT-PCR), consistent with one or more exemplary embodiments of the present disclosure. As shown in RNA expression profile 200, Group A refers to an exemplary transformed Pseudomonas aeruginosa bacteria grown for 4 h in the presence of imipenem, Group B refers to an exemplary transformed Pseudomonas aeruginosa bacteria grown for 18 h in the presence of imipenem, and Group C refers to an exemplary transformed Pseudomonas aeruginosa bacteria grown for 36 h in the presence of imipenem. RNA expression profile 200 demonstrates that an exemplary siDNA may significantly reduce the level of blaIMP-1 mRNA, compared to an exemplary untreated Pseudomonas aeruginosa (as negative control). Fold change of an exemplary blaIMP-1 mRNA in untreated Pseudomonas aeruginosa, Group A, Group B, and Group C—shown in RNA expression profile 200—were about 1, 0.088, 0.020, and 0.27, respectively. In other words, RNA expression profile 200 shows that gene expression in Group A, Group B, and Group C, may decrease by 11.36, 50, and 3.7 folds, compared to an exemplary untreated Pseudomonas aeruginosa. Thus, it may be concluded that Pseudomonas aeruginosa carrying an exemplary siDNA may exhibit a significant sensitivity to imipenem, compared to an exemplary untreated Pseudomonas aeruginosa.

On the other hand, to further confirm success of an exemplary siDNA in silencing an exemplary blaIMP-1 gene in Pseudomonas aeruginosa, minimum inhibitory concentration (MIC) of imipenem was determined in an exemplary transformed Pseudomonas aeruginosa. To this end, MICs of imipenem that may inhibit the growth of an exemplary transformed Pseudomonas aeruginosa (with an exemplary siDNA) was determined after 1-4 h, 2-18 h, and 3-36 h, by broth dilution method performed in a 96-well microtiter plate. Exemplary MICs of imipenem to inhibit growth of the transformed Pseudomonas aeruginosa was compared to an exemplary negative control/untreated Pseudomonas aeruginosa (i.e., to MICs of imipenem that may inhibit the growth of an exemplary untransformed Pseudomonas aeruginosa).

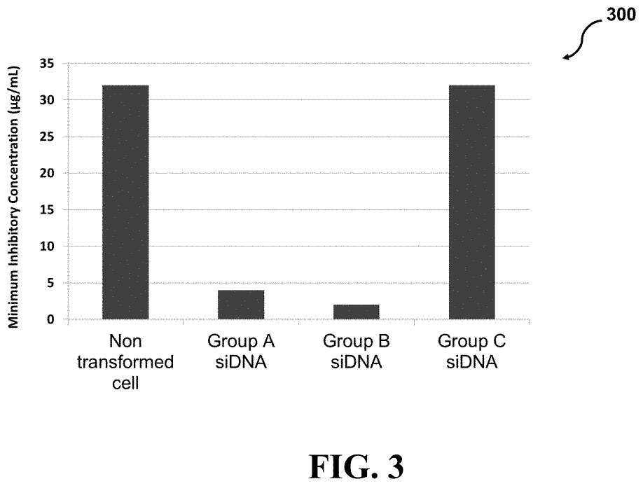

To conduct an exemplary MIC assay, bacterial culture grown to log phase was adjusted to about 5×10⁵ (cells/mL) in Muller-Hinton broth. Then, the antimicrobial effect of imipenem on an exemplary transformed Pseudomonas aeruginosa and an exemplary untreated Pseudomonas aeruginosa was determined by measuring turbidity of bacterial growth in each well, using a microtiter plate reader. FIG. 3 illustrates imipenem MIC assay 300 for an exemplary transformed Pseudomonas aeruginosa and an exemplary untreated Pseudomonas aeruginosa after 4, 18, and 36 h, consistent with one or more exemplary embodiments of the present disclosure. Referring to imipenem MIC assay 300, Group A refers to an exemplary transformed Pseudomonas aeruginosa grown for 4 h in the presence of imipenem, Group B refers to an exemplary transformed Pseudomonas aeruginosa grown for 18 h in the presence of imipenem, and Group C refers to an exemplary transformed Pseudomonas aeruginosa grown for 36 h in the presence of imipenem. Based on imipenem MIC assay 300, Group A and B were sensitive to 2 μg/mL and 4 μg/mL imipenem, respectively (low MICs). However, Group C demonstrated a significantly high MIC (32 μg/mL) similar to an exemplary negative control (i.e., an exemplary untreated Pseudomonas aeruginosa).

In addition to conducting an exemplary MIC assay, sensitivity of an exemplary transformed Pseudomonas aeruginosa and an exemplary untreated Pseudomonas aeruginosa (negative control) to imipenem and kanamycin was determined by E-test and disc diffusion susceptibility testing. Thereby, an exemplary suspension for both of exemplary transformed and untreated Pseudomonas aeruginosa, with an optical density equivalent to McFarland turbidity standard of 0.5, were prepared. Then, about 10 μL of exemplary suspensions were inoculated onto Mueller-Hinton agar plates (with no antibiotics added to Mueller-Hinton agar). E-test strips and/or imipenem antimicrobial susceptibility discs were gently laid on the surface of Mueller-Hinton agar. Plates were assessed after about 4 h, 18 h, and 36 h of incubation at 37° C. under aerobic conditions.

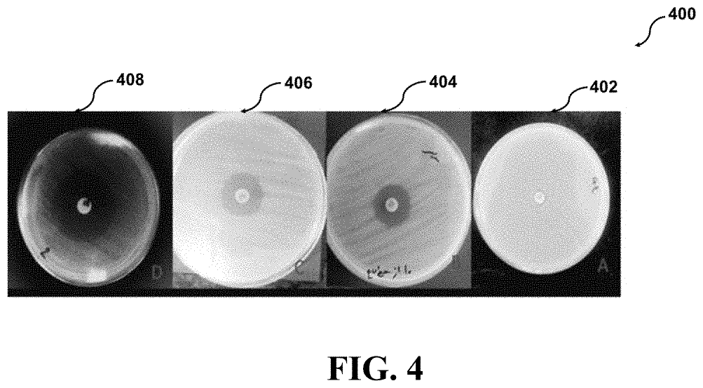

FIG. 4 illustrates disc diffusion susceptibility testing 400 for an exemplary transformed Pseudomonas aeruginosa and an exemplary untreated Pseudomonas aeruginosa (negative control) in the presence of an imipenem disc, consistent with one or more exemplary embodiments of the present disclosure. As shown in FIG. 4, plate 402 shows disc diffusion susceptibility testing on an exemplary untreated Pseudomonas aeruginosa. Plate 402 shows that the untreated Pseudomonas aeruginosa was resistant to imipenem (no growth inhibition zone was observed around the imipenem antimicrobial susceptibility disc). In contrast, plate 404, plate 406, and plate 408 show disc diffusion susceptibility testing on an exemplary transformed Pseudomonas aeruginosa. As shown in plate 404, plate 406, and plate 408, Pseudomonas aeruginosa carrying an exemplary siDNA exhibited a significant sensitivity to imipenem (growth inhibition zones with different diameters ranging from 8 mm to 23 mm were observed).

### Example 4: Evaluating the Effect of an Exemplary siDNA In Vivo

In this example, the effect of an exemplary siDNA on an exemplary imipenem-resistant Pseudomonas aeruginosa was assessed in 40 mice with chronic Pseudomonas aeruginosa lung infection. For this purpose, a 1% w/v concentration of chitosan polymer was prepared, followed by preparing two different ratios of chitosan 1% w/v to an exemplary siDNA—in phosphate buffered saline (PBS)—including an exemplary 1:1 ration and an exemplary 0.5:1 ratio.

A chronic Pseudomonas aeruginosa lung infection model (using imipenem-resistant Pseudomonas aeruginosa) was established in 40 mice (25-30 g). Exemplary mice were randomly divided into three groups (each group comprising 10 mice) including: i) an exemplary control group that only received imipenem after acquiring chronic infection, ii) an exemplary first group that received imipenem in combination with an exemplary siDNA (ratio 0.5:1 of chitosan 1% w/v to an exemplary siDNA) after acquiring chronic infection, and iii) an exemplary second group that received imipenem in combination with an exemplary siDNA (ratio 1:1 of chitosan 1% w/v to an exemplary siDNA) after acquiring chronic infection. After 3 days of treating each group (i.e., control group, first group, and second group) with exemplary medicaments set forth in (i), (ii), and (iii), lung tissue was excised to be examined histologically. A portion of the excised tissues was suspended in paraffin and processed for assessment by hematoxylin and eosin staining. The level of tissue damage in an exemplary control group, first group, and second group was observed and analyzed by light microscopy.

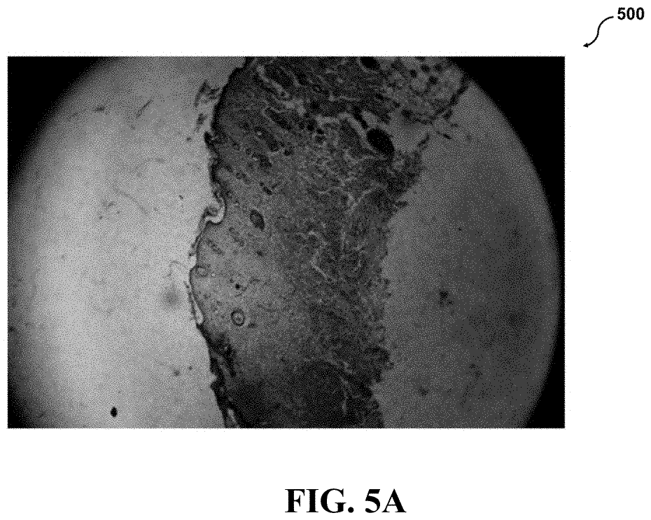

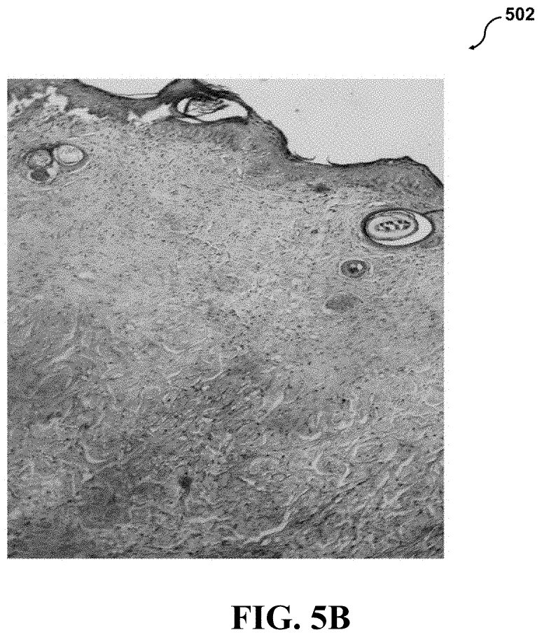

FIG. 5A illustrates histological images 500 of an exemplary excised lung tissue from an exemplary infected mouse with imipenem-resistant Pseudomonas aeruginosa which received neither of imipenem and/or an exemplary siDNA, consistent with one or more exemplary embodiments of the present disclosure. FIG. 5B illustrates histological images 502 of an exemplary excised lung tissue from an exemplary infected mouse with imipenem-resistant Pseudomonas aeruginosa which received imipenem in combination with an exemplary siDNA, consistent with one or more exemplary embodiments of the present disclosure. Histological images 500 show an obvious edema and damage in the lung tissue of an exemplary infected mouse with imipenem-resistant Pseudomonas aeruginosa which received neither of imipenem and/or an exemplary siDNA. Notwithstanding, as shown in histological images 502, a significantly less edema and tissue damage was observed in the lung tissue of an exemplary infected mouse with imipenem-resistant Pseudomonas aeruginosa which received imipenem in combination with the siDNA.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. An element proceeded by “a” or “an” does not, without further constraints, preclude the existence of additional identical elements in the process, method, article, or apparatus that comprises the element.

Unless otherwise stated, all measurements, values, ratings, positions, magnitudes, sizes, and other specifications that are set forth in this specification, are approximate, not exact. They are intended to have a reasonable range that is consistent with the functions to which they relate and with what is customary in the art to which they pertain.

It will be understood that the terms and expressions used herein have the ordinary meaning as is accorded to such terms and expressions with respect to their corresponding respective areas of inquiry and study, except where specific meanings have otherwise been set forth herein. Relational terms such as “first” and “second” and the like may be used solely to distinguish one entity or action from another without necessarily requiring or implying any actual such relationship or order between such entities or actions.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it may be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A small interfering deoxyribonucleic acid (siDNA), comprising:
a sense strand comprising the nucleic acid sequence set forth in SEQ ID NO: 1; and
an antisense strand, complementary to the sense strand, the antisense strand comprising the nucleic acid sequence set forth in SEQ ID NO: 2.

2. The siDNA of claim 1, wherein the antisense strand is capable of hybridizing to a segment of a coding strand of class 1 integron metallo-beta-lactamase (blaIMP-1) gene.

3. The siDNA of claim 2, wherein the coding strand of the blaIMP-1 gene comprises the nucleic acid sequence set forth in SEQ ID NO: 3.

4. The siDNA of claim 2, wherein the segment of the coding strand of the blaIMP-1 gene comprises nucleotide 302 to nucleotide 325 of SEQ ID NO: 3.

5. The siDNA of claim 1, wherein the sense strand is capable of hybridizing to a segment of a non-coding strand of class 1 integron metallo-beta-lactamase (blaIMP-1) gene.

6. The siDNA of claim 5, wherein the non-coding strand of the blaIMP-1 gene comprises the nucleic acid sequence set forth in SEQ ID NO: 4.

7. The siDNA of claim 5, wherein the segment of the non-coding strand of the blaIMP-1 gene comprises nucleotide 417 to nucleotide 440 of SEQ ID NO: 4.

8. The siDNA of claim 1, wherein the sense strand comprises a protospacer adjacent motif (PAM).

9. The siDNA of claim 8, wherein the PAM comprises Cytosine 2 and Cytosine 3 (C2-C3) of SEQ ID NO: 1.

10. The siDNA of claim 1, wherein the sense strand comprises a 6-Carboxyfluorescein (6-FAM) at the 5′ end of the sense strand.

11. The siDNA of claim 1, wherein the sense strand comprises a tetramethylrhodamine (TAMARA) dye at the 3′ end of the sense strand.

12. The siDNA of claim 1, wherein the antisense strand comprises a 6-Carboxyfluorescein (6-FAM) at the 5′ end of the antisense strand.

13. The siDNA of claim 1, wherein the antisense strand comprises a tetramethylrhodamine (TAMARA) at the 3′ end of the antisense strand.

14. A small interfering deoxyribonucleic acid (siDNA), comprising:
a sense strand comprising the nucleic acid sequence set forth in SEQ ID NO: 1, the sense strand capable of hybridizing to a segment of a non-coding strand of class 1 integron metallo-beta-lactamase (blaIMP-1) gene; and
an antisense strand, complementary to the sense strand, the antisense strand comprising the nucleic acid sequence set forth in SEQ ID NO: 2, wherein the antisense strand is capable of hybridizing to a segment of a coding strand of the blaIMP-1 gene.

15. The siDNA of claim 14, wherein the coding strand of the blaIMP-1 gene comprises the nucleic acid sequence set forth in SEQ ID NO: 3.

16. The siDNA of claim 14, wherein the segment of the coding strand of the blaIMP-1 gene comprises nucleotide 302 to nucleotide 325 of SEQ ID NO: 3.

17. The siDNA of claim 14, wherein the non-coding strand of the blaIMP-1 gene comprises the nucleic acid sequence set forth in SEQ ID NO: 4.

18. The siDNA of claim 14, wherein the segment of the non-coding strand of the blaIMP-1 gene comprises nucleotide 417 to nucleotide 440 of SEQ ID NO: 4.

19. The siDNA of claim 14, wherein the sense strand comprises a protospacer adjacent motif (PAM).

20. The siDNA of claim 19, wherein the PAM comprises Cytosine 2 and Cytosine 3 (C2-C3) of SEQ ID NO: 1.


SEQUENCE LISTING (5 sequences)

SEQ ID NO: 1 - DNA, 28 bp, synthetic construct
  source 1..28: mol_type = other DNA
  iDNA 1..28: note = sense strand
gccgacatcgccttcggcgtttatgatt

SEQ ID NO: 2 - DNA, 28 bp, synthetic construct
  iDNA 1..28: note = antisense strand
  source 1..28: mol_type = other DNA
aacgcgagaaagtatgaacctagcactt

SEQ ID NO: 3 - DNA, 741 bp, Acinetobacter baumannii
  source 1..741: mol_type = genomic DNA
atgaaaaaattatttgttttatgtgtattcttcttctgcaacattgcagttgcagaagaatctttgcctgatttaaaaattgagaagcttgaagaaggcgtttatgttcatacttcgtttgaagaagttaaaggttggagtgtggtcactaaacacggtttggtggttcttgtgaaaaatgacgcctatctgattgatactccaattactgctaaagatactgaaaaattagtcaattggtttgttgagcggggctataaaatcaaaggcagtatttccacacatttccatggtgacagtacggctggaatagagtggcttaattctcaatctatccccacatatgcttctgaattaacaaatgaacttcttaaaaaagacaataaggtacaagctaaacactcttttaatggggttagttattcactaattaaaaacaaaattgaagttttttatccaggcccagggcacactcaagataacgtagtggtttggttacctgaaaagaaaattttattcggtggttgctttgttaaaccggacggtcttggctatttgggggacgcaaatttagaagcttggccaaagtccgctaaaatattaatgtctaaatatggtaaagcaaaactagttgtgtcgagtcatagtgatattggagatgtatcactcttgaaacgtacatgggagcaggctgttaaagggctgaatgaaagtaaaaaatcatcacagccaagcgactaa

SEQ ID NO: 4 - DNA, 741 bp, Acinetobacter baumannii
  source 1..741: mol_type = genomic DNA
ttagtcgcttggctgtgatgattttttactttcattcagccctttaacagcctgctcccatgtacgtttcaagagtgatacatctccaatatcactatgactcgacacaactagttttgctttaccatatttagacattaatattttagcggactttggccaagcttctaaatttgcgtcccccaaatagccaagaccgtccggtttaacaaagcaaccaccgaataaaattttcttttcaggtaaccaaaccactacgttatcttgagtgtgccctgggcctggataaaaaacttcaattttgtttttaattagtgaataactaaccccattaaaagagtgtttagcttgtaccttattgtcttttttaagaagttcatttgttaattcagaagcatatgtggggatagattgagaattaagccactctattccagccgtactgtcaccatggaaatgtgtggaaatactgcctttgattttatagccccgctcaacaaaccaattgactaatttttcagtatctttagcagtaattggagtatcaatcagataggcgtcatttttcacaagaaccaccaaaccgtgtttagtgaccacactccaacctttaacttcttcaaacgaagtatgaacataaacgccttcttcaagcttctcaatttttaaatcaggcaaagattcttctgcaactgcaatgttgcagaagaagaatacacataaaacaaataattttttcat

SEQ ID NO: 5 - protein, 246 aa, Acinetobacter baumannii
MKKLFVLCVFFFCNIAVAEESLPDLKIEKLEEGVYVHTSFEEVKGWSVVTKHGLVVLVKNDAYLIDTPITAKDTEKLVNWFVERGYKIKGSISTHFHGDSTAGIEWLNSQSIPTYASELTNELLKKDNKVQAKHSFNGVSYSLIKNKIEVFYPGPGHTQDNVVVWLPEKKILFGGCFVKPDGLGYLGDANLEAWPKSAKILMSKYGKAKLVVSSHSDIGDVSLLKRTWEQAVKGLNESKKSSQPSD